(19) United States
(12) Patent Application Publication
(10) Pub. No.: US 2025/0120874 A1
(43) Pub. Date: Apr. 17, 2025

(54) TEMPERATURE HYDROTHERAPY MUMMY BAG, HYDROTHERAPY BAG, HYDROTHERAPY BED, OR HYDROTHERAPY BLANKET FOR DRY-HYDROTHERAPY BODY MASSAGE

(71) Applicant: Keith Elam, Eugene, OR (US)

(72) Inventor: Keith Elam, Eugene, OR (US)

(21) Appl. No.: 18/486,287

(22) Filed: Oct. 13, 2023

(51) Int. Cl. A61H 9/00; A61F 7/00; A61F 7/02; A61H 23/02; A61N 1/04

(52) U.S. Cl. CPC A61H 9/0021; A61F 7/0085; A61F 7/02; A61H 23/02; A61F 2007/0054; A61H 2009/0042; A61H 2201/0157; A61H 2201/107; A61H 2201/1238; A61H 2201/5051; A61H 2201/5058; A61H 2205/06; A61H 2205/081; A61H 2205/10; A61N 1/0456

(57) ABSTRACT

The invention discloses a multilayered Hydrotherapy Blanket, Hydrotherapy Mummy Bag, and Hydrotherapy Bag that has an outer layer, and when compressed with air or water, this can be wrapped around the body tightly, while the inner layer circulates high pressured hot or cold water around the body. Compressed air is injected into Hydrotherapy tubes that have nozzle holes that release the air mixed with hot or cold high-pressure water onto a thin layer of water-proof barrier that is touching the body, simulating a Jacuzzi Hydrotherapy effect, without the body getting wet. The hot or cold water can be pumped from the top of the Hydrotherapy Mummy Bag, Hydrotherapy Bag, Hydrotherapy Blanket and Hydrotherapy Bed, then the hot or cold water can be pumped in the opposite direction inside Hydrotherapy tubes, causing an inward-outward Hydrotherapy massage effect. One can lay inside a Hydrotherapy Mummy Bag to get a full body massage. The invention comprises moving magnets to create a moving magnetic field to travel over the body. It allows the magnetic fabric to be used to help disperse the magnetic field around the body. The claimed invention also allows a Transcutaneous Electrical Nerve Stimulation (TENS) device configured to be attached to the said Hydrotherapy Mummy Bag, Hydrotherapy Bag, Hydrotherapy Blanket and Hydrotherapy Bed to help stimulate the muscles and nerves. The claimed invention also allows the portable Hydrotherapy Machine to be transported in a small vehicle for home to home Dry-hydrotherapy. The claimed invention also allows the portable Hydrotherapy Machine to operate on at least 10 gallons of hot or cold water/contaminated water to provide a safe Dry-hydrotherapy massage to the arm, leg or back.

### FIELD OF THE INVENTION

The present invention relates generally to an universal Hydrotherapy devices like a Hydrotherapy tub, Jacuzzi, whirlpool, and hot tub, and more particularly to applying a massaging effect to the body of a user utilizing high-pressure hot and cold water. It further relates to air mattresses and water beds. The invention further relates to a TENS Device, (Transcutaneous Electrical Nerve Stimulation). It further relates to an oxygen therapy devices.

### BACKGROUND OF THE INVENTION

Background description includes information that may be useful in understanding the present invention. It is not an admission that any of the information provided herein is prior art or relevant to the presently claimed invention, or that any publication specifically or implicitly referenced is prior art.

Massage is a time-honored and generally effective therapy for muscular injuries, strains and general soreness but, although massage is still recommended by many physicians for such purposes, this therapy is largely unavailable due to a scarcity of trained, qualified masseurs. As a result, many devices and apparatus have been proposed in the past for producing a massage-like manipulation of a user's body by mechanically or electrically-generated vibrations or pulsations, usually accompanied by heating. In addition, massage-stimulative devices utilizing pulsations of pressurized water have recently become popular, such as pulsation-type shower heads, whirlpool baths, and the like.

Various types of massage units have been provided heretofore and generally encompass mechanical and electrical hand-held units that vibrate, pulsate, and provide heat. Also known in the art are shower heads arranged to expel water in a pulsating fashion whereby a person taking a shower is treated to a variety of sprays which can be physically directed at will to any desired spot on the body.

Also, there are known devices that provides heated water circulated to a flexible bag positioned on a person's back, for example. The action of the circulating water through the bag, and the heat emanating therefrom, serve to provide therapy for a muscle injury or the like.

Hot tubs have been used for therapy for thousands of years to heal the body from injuries. The Jacuzzi was invented to apply pressured hot water and air to the body parts, but the Jacuzzi is made from either wood, cement, fiberglass, or stainless steel, and sometimes having to use a crane to lift and move a Hydrotherapy tub, Jacuzzi, whirlpool, or hot tub. Another problem with these types of Jacuzzis, whirlpools, or hot tubs is that chlorine or other chemicals must be used to purify the water. There are Jacuzzis, whirlpools, or hot tubs that use UV lights to purify the water, but still, any therapeutic office or hospital has to change the water before each use, to prevent body injuries from being infected. It also takes a lot of heat or energy to heat up the water in any Hydrotherapy tub, Jacuzzi, whirlpool, or hot tub on Earth. All Hydrotherapy tubs, Jacuzzis, whirlpools, or hot tubs can not use dirty water or contaminated water, without resulting in damage to the Jacuzzi equipment or injury or even possible death in some cases, when soaking in hazardous water for hours at a time. many hydrotherapy tubs, Jacuzzis, whirlpools, or hot tubs have to use AC to operate with, and many Nations cannot afford to pay a therapeutic Jacuzzi which costs more than what one makes in one year. There is also a major issue for some Nations around the World who do not have 100 gallons or more of bath water available to fill up a Hydrotherapy tub, Jacuzzi, whirlpool, or hot tub. Another issue with Hydrotherapy tubs, Jacuzzis, whirlpools, or hot tubs, you can only use either hot or cold water at one time, you can not use both hot and cold water inside a Hydrotherapy tub, Jacuzzi, whirlpool, or hot tub at the same time, to be able to control the body temperature from becoming too hot or too cold.

In recent years, the emergence of deadly viruses with contagion properties is spelling havoc in our societies, resulting in severe straining of the healthcare systems as also of the economies. Therapists around the World use a hot water Jacuzzi for therapy to repair and heal body parts. When a foot, leg, hand or arm, or body has to have this type of therapy, big stainless steel Hydrotherapy tubs are used for Hydrotherapy.

In view of the foregoing, there is a need for a hot water Jacuzzi for therapy that consumes less water, less energy to heat water, occupies less space, and portable to carry and use inside a vehicle for home-to-home Hydrotherapy. Also, there is a need for a hot water Jacuzzi for providing more effective and economical Dry-hydrotherapy body massage for healing body parts.

The present application provides these and other advantages as will be apparent from the following detailed description and accompanying figures.

### SUMMARY OF THE INVENTION

This summary is provided to introduce a selection of concepts in a simplified format that are further described in the detailed description of the present disclosure. This summary is not intended to identify key or essential inventive concepts of the present disclosure, nor is it intended for determining the scope of the present disclosure.

It is an object of the invention to provide an all-temperature Hydrotherapy Mummy Bag, Hydrotherapy Bag, Hydrotherapy Bed, or Hydrotherapy Blanket for Dry-hydrotherapy body massage that consumes less water, less energy to heat water, occupies less space, and is portable to carry and use inside a vehicle for home-to-home Dry-hydrotherapy.

It is another object of the invention to provide a cold and hot water Dry-hydrotherapy for providing more effective and economical Dry-hydrotherapy body massage for healing body injuries.

It is another object of the invention to provide cold and hot water Dry-hydrotherapy that uses water pressure for moving the magnetic ball bearings through Magnetic tubes or Ceramic Silicon Nitride ball bearings through Hydrotherapy tubes to help healing the body injuries.

It is another object of the invention, a compressed air is injected into Hydrotherapy tubes that have nozzle holes that release in the air mixed with hot or cold high-pressure water onto a thin layer of water-proof barrier that is touching the body, simulating a Jacuzzi effect, without the body getting wet.

It is another object of the invention that the Hydrotherapy Machine is portable and able to operate on DC Batteries, DC Solar Panels, or portable Wind Turbine Generators.

It is another object of the invention that the Hydrotherapy Machine is portable and able to run on at least 10 gallons of water/contaminated water to provide a safe Dry-hydrotherapy massage on the leg, arm or back.

According to an embodiment of the present disclosure, provides a multilayered Hydrotherapy Bag, Hydrotherapy Blanket or Hydrotherapy Mummy Bag that has an outer layer, and when compressed with air or water, the Hydrotherapy Bag, Hydrotherapy Blanket or

Hydrotherapy Mummy Bag can wrap around the body tightly, while the inner layer of the Hydrotherapy Bag, Hydrotherapy Blanket or Hydrotherapy Mummy Bag circulates high pressured hot or cold water around the body, as compressed air is being injected into the hot or cold water to provide a Jacuzzi effect. The hot or cold water can be pumped from the top of the Hydrotherapy Mummy Bag, Hydrotherapy Bag, Hydrotherapy Blanket or Hydrotherapy Bed to the bottom of the Hydrotherapy Mummy Bag, Hydrotherapy Bag, Hydrotherapy Blanket or Hydrotherapy Bed then the hot or cold water can be forced back up the opposite direction of the plurality of Hydrotherapy tubes, causing an inward-outward massage effect.

The disclosure comprises a discharge pump that is used to pump the water out from the Hydrotherapy Mummy Bag, Hydrotherapy Bag, Hydrotherapy Blanket or Hydrotherapy Bed back into the water tanks.

The disclosure comprises moving magnets to create a moving magnetic field to travel over the body. It allows the magnetic fabric to be used to help disperse the magnetic field around the body. A Silicone or other Waterproof material used as a waterproof seal on Copper or Silver Infused Conductive Fabric, the Copper or Silver Infused Conductive Fabric used to help increase a Transcutaneous Electrical Nerve Stimulation (TENS) device range. Further, it discloses the use of Oxygen which is produced in an Oxygen/Hydrogen Gas Chambers in order for injecting Oxygen into the Hydrotherapy Mummy Bag, Hydrotherapy Bag, Hydrotherapy Bed, or Hydrotherapy Blanket to help heal the wounds of the user.

To further clarify the advantages and features of the present disclosure, a more particular description of the disclosure will be rendered by reference to specific embodiments thereof, which is illustrated in the appended drawings. It is appreciated that these drawings depict only typical embodiments of the disclosure and are therefore not to be considered limiting of its scope. The disclosure will be described and explained with additional specificity and detail with the accompanying drawings.

### BRIEF DESCRIPTION OF THE DRAWINGS

The subject matter that is regarded as the invention is particularly pointed out and distinctly claimed in the claims at the conclusion of the specification. The foregoing and other aspects, features, and advantages of the invention are apparent from the following detailed description taken in conjunction with the accompanying drawings in which:

FIG. 1 illustrates schematics inside view of the portable Dry-hydrotherapy system, in accordance with an embodiment of the present disclosure.

FIG. 2 illustrates the Hydrotherapy Bag, Hydrotherapy Mummy Bag, Hydrotherapy Bed, or Hydrotherapy Blanket, in accordance with an embodiment of the present disclosure.

FIG. 3 illustrates the Portable Hydrotherapy Machine, in accordance with an embodiment of the present disclosure.

FIG. 4 illustrates the Hydrotherapy Pump System, in accordance with an embodiment of the present disclosure.

FIG. 5 illustrates an air compressor, in accordance with an embodiment of the present disclosure.

FIG. 6 illustrates the Hydrotherapy Bag, Hydrotherapy Mummy Bag, Hydrotherapy Bed, or Hydrotherapy Blanket, in accordance with an embodiment of the present disclosure.

FIG. 7 illustrates the Hot and Cold Water Hydrotherapy Machine for Dry-hydrotherapy body massage, in accordance with an embodiment of the present disclosure.

Further, skilled artisans will appreciate that elements in the drawings are illustrated for simplicity and may not have necessarily been drawn to scale. Furthermore, in terms of the construction of the device, one or more components of the device may have been represented in the drawings by conventional symbols, and the drawings may show only those specific details that are pertinent to understanding the embodiments of the present invention so as not to obscure the drawings with details that will be readily apparent to those of ordinary skill in the art having benefit of the description herein.

### DETAILED DESCRIPTION OF THE INVENTION

For the purpose of promoting an understanding of the principles of the invention, reference will now be made to the embodiment illustrated in the drawings, and specific language will be used to describe the same. It will nevertheless be understood that no limitation of the scope of the invention is thereby intended. Such alterations and further modifications in the illustrated system, and such further applications of the principles of the invention as illustrated therein would be contemplated as would normally occur to one skilled in the art to which the invention relates. Unless otherwise defined, all technical and scientific terms used herein have the same meaning as commonly understood by one of ordinary skilled in the art. The system, methods, and examples provided herein are illustrative only and are not intended to be limiting.

The term “some” as used herein is to be understood as “none or one or more than one or all.” Accordingly, the terms “none,” “one,” “more than one,” “more than one, but not all” or “all” would all fall under the definition of “some.” The term “some embodiments” may refer to no embodiments or to one embodiment or to several embodiments or to all embodiments, without departing from the scope of the present disclosure.

The terminology and structure employed herein is for describing, teaching, and illuminating some embodiments and their specific features. It does not in any way limit, restrict or reduce the spirit and scope of the claims or their equivalents.

More specifically, any terms used herein such as but not limited to “includes,” “comprises,” “has,” “consists,” and grammatical variants thereof do not specify an exact limitation or restriction and certainly do not exclude the possible addition of one or more features or elements, unless otherwise stated, and furthermore must not be taken to exclude the possible removal of one or more of the listed features and elements, unless otherwise stated with the limiting language “must comprise” or “needs to include.”

Whether or not a certain feature or element was limited to being used only once, either way, it may still be referred to as “one or more features” or “one or more elements” or “at least one feature” or “at least one element.” Furthermore, the use of the terms “one or more” or “at least one” feature or element do not preclude there being none of that feature or element, unless otherwise specified by limiting language such as “there needs to be one or more” or “one or more element is required.”

Unless otherwise defined, all terms, and especially any technical and/or scientific terms, used herein may be taken to have the same meaning as commonly understood by one having ordinary skills in the art.

Reference is made herein to some “embodiments.” It should be understood that an embodiment is an example of a possible implementation of any features and/or elements presented in the attached claims. Some embodiments have been described for the purpose of illuminating one or more of the potential ways in which the specific features and/or elements of the attached claims fulfill the requirements of uniqueness, utility and non-obviousness.

Use of the phrases and/or terms including, but not limited to, “a first embodiment,” “a further embodiment,” “an alternate embodiment,” “one embodiment,” “an embodiment,” “multiple embodiments,” “some embodiments,” “other embodiments,” “further embodiment”, “furthermore embodiment”, “additional embodiment” or variants thereof do not necessarily refer to the same embodiments. Unless otherwise specified, one or more particular features and/or elements described in connection with one or more embodiments may be found in one embodiment, or may be found in more than one embodiment, or may be found in all embodiments, or may be found in no embodiments. Although one or more features and/or elements may be described herein in the context of only a single embodiment, or alternatively in the context of more than one embodiment, or further alternatively in the context of all embodiments, the features and/or elements may instead be provided separately or in any appropriate combination or not at all. Conversely, any features and/or elements described in the context of separate embodiments may alternatively be realized as existing together in the context of a single embodiment.

Any particular and all details set forth herein are used in the context of some embodiments and therefore should not be necessarily taken as limiting factors to the attached claims. The attached claims and their legal equivalents can be realized in the context of embodiments other than the ones used as illustrative examples in the description below.

Embodiments of the present invention will be described below in detail with reference to the accompanying drawings.

The present invention provides a multilayered Hydrotherapy Mummy Bag, Hydrotherapy Bag and Hydrotherapy Blanket that has an outer layer, and when compressed or pumped with air or water, the Hydrotherapy Mummy Bag, Hydrotherapy Bag and Hydrotherapy Blanket can wrap around the body tightly, while the inner layer of the Hydrotherapy Mummy Bag, Hydrotherapy Bag and Hydrotherapy Blanket circulates high pressured hot or cold water around the body. In the present Invention the Hydrotherapy Bed, Hydrotherapy Bag, Hydrotherapy Blanket and Hydrotherapy Mummy Bag where high PSI hot or cold water are injected into Hydrotherapy tubes and Nozzles onto the inner layer of the Hydrotherapy Bed, Hydrotherapy Bag, Hydrotherapy Blanket and Hydrotherapy Mummy Bag to provide a Jacuzzi like Hydrotherapy effect upon the body.

In the present disclosure, compressed air is pumped with water into Hydrotherapy tubes that have nozzle holes that release the air mixed with hot or cold high-pressure water onto a thin layer of water-proof barrier that is touching the body, simulating a Jacuzzi like Hydrotherapy effect, without the body getting wet. The invention comprises moving magnets to create a moving magnetic field to travel over the body. It allows a magnetic fabric to be used to help disperse the magnetic field around the body. A Copper or Silver Infused Conductive Fabric helps increase a Transcutaneous Electrical Nerve Stimulation (TENS) device range. Further, it discloses the use of Oxygen which is produced in Oxygen/Hydrogen Gas Chambers in order to inject Oxygen into the Hydrotherapy Mummy Bag, Hydrotherapy Bag, Hydrotherapy Bed, or Hydrotherapy Blanket to help heal the wounds of the user.

In an embodiment of the present invention, a multilayered Hydrotherapy Mummy Bag, Hydrotherapy Blanket, Hydrotherapy Bag or Hydrotherapy Bed for Dry-hydrotherapy comprises at least three or more layers of waterproof fabrics, at least one cold water storage tank and at least one hot water storage tank configured to store cold and hot water respectively, for circulating inside the Hydrotherapy Blanket, Hydrotherapy Bag, Hydrotherapy Mummy Bag or the Hydrotherapy Bed, at least one or more AC/DC water pumps to circulate the hot or cold water inside the Hydrotherapy Blanket, Hydrotherapy Bag, Hydrotherapy Mummy Bag or the Hydrotherapy Bed and into the hot or cold water tanks, at least one insulated hot water inlet tubing or pipe configured to separately circulate the hot water inside the Hydrotherapy Blanket, Hydrotherapy Bag, Hydrotherapy Mummy Bag or the Hydrotherapy Bed, at least one outlet line from the AC/DC water pump configured to circulate the hot and cold water inside the Hydrotherapy Blanket, Hydrotherapy Bag, Hydrotherapy Mummy Bag or the Hydrotherapy Bed, at least one AC/DC 2-3-Way Solenoid Valve or 2-3-Way Motorized Valve configured to circulate hot or cold water through the AC/DC water pump, cold water from the at least one cold water storage tank or hot water from the at least one hot water storage tank, into the Hydrotherapy Blanket, Hydrotherapy Bag, Hydrotherapy Mummy Bag or Hydrotherapy Bed, at least one inlet water line for the Solenoid Valve Valve or Motorized Valve, at least one outlet water line for the three-way Solenoid Valve or motorized three-way valve attached to at least one water pump manifold, at least one AC/DC water drain pump attached to at least one drain hose to remove the water from the Hydrotherapy Blanket, Hydrotherapy Bag, Hydrotherapy Mummy Bag or Hydrotherapy Bed back into at least one water storage tank, at least one air inlet line to an outer liner configured to seal the Hydrotherapy Bag, Hydrotherapy Mummy Bag and Hydrotherapy Blanket tightly around body of the user, at least one air hose configured to attach to the Hydrotherapy Blanket, Hydrotherapy Bag, Hydrotherapy Mummy Bag or Hydrotherapy Bed, at least one AC/DC air compressor configured to compress air into the hot or cold water inside the Hydrotherapy Blanket, Hydrotherapy Bag, Hydrotherapy Mummy Bag or Hydrotherapy Bed, plurality of Magnetic tubes configured to accommodate plurality of magnetic ball bearings, plurality of Hydrotherapy tubes configured to accommodate plurality of Ceramic Silicon Nitride ball bearings for providing massage, and plurality of plastic ball bearing separator configured to separate the magnetic field and to prevent sticking of magnetic bearings, when two or more magnetic bearings are used to increase the magnetic field inside the plurality of magnetic tubes. The multilayered Hydrotherapy Bag, Hydrotherapy Mummy Bag, Hydrotherapy Bed, or Hydrotherapy Blanket comprises a Transcutaneous Electrical Nerve Stimulation (TENS) device configured to stimulate the muscles and nerves, and the waterproofing element is Silicone or other Waterproof material configured to waterproof a Copper or Silver Infused Conductive Fabric, the Copper or Silver Infused Conductive Fabric to enhance the TENS device range. The TENS device comprises positive and negative wire connections that are embedded into the Copper or Silver Infused Conductive Fabric.

The multilayered Hydrotherapy Bag. Hydrotherapy Mummy Bag, Hydrotherapy Bed, or Hydrotherapy Blanket further comprises DC supply Converters for AC to DC configured to supply DC Volts to the Hydrotherapy Machine's DC electrical components, a positive DC wire that goes to a Cathode, and a negative DC wire that goes to an Anode, at least one Oxygen/Hydrogen Gas Chambers configured to hold water and an Electrolyte to produce pure Oxygen using the Anode for circulating the pure Oxygen inside the Hydrotherapy Blanket, Hydrotherapy Bag, Hydrotherapy Mummy Bag or Hydrotherapy Bed via at least one Oxygen line going from an Oxygen separator tube containing Oxygen extracted from H₂O, plurality of Hydrotherapy tubes configured to allow high pressured air and water to travel out of the Hydrotherapy tubes to massage the user's body without having the water touching the skin. The Hydrotherapy nozzle holes configured to allow high-pressure air and water to be injected from the Hydrotherapy tubes onto the user's body to simulate a Hydro-therapeutic effect upon the skin and muscles of the user. The plurality of magnetic ball bearings inside the plurality of Magnetic tubes provides a magnetic field as the plurality of magnetic ball bearings rolls down the Magnetic tubes, the magnetic field travels across the user's body.

In an embodiment of the present disclosure, material for at least three or more layers of the Hydrotherapy Mummy Bag, Hydrotherapy Bag, Hydrotherapy Bed, or Hydrotherapy Blanket is selected from either Leather, Hemp fabric, waterproof Hemp fabric, waterproof Cotton fabric, waterproof Nylon, Neoprene, or other waterproof materials capable of withstanding high temperature and Pound per Square Inch (PSI).

In an embodiment of the present disclosure, at least one AC/DC water heating element is configured to heat the water in at least one hot water storage tank.

In an embodiment of the present disclosure, an Electrical storage box mounted on the at least one water storage tank for protecting at least one or more relay switches, timers, breakers, AC to DC Converters configured to control the relay switches, timers, breakers, etc., to operate the opening and closing of the Solenoid Valves or Motorized Valves, operating the heater elements and water pumps.

In an embodiment of the present disclosure, an air compressor water drain valve is configured to drain the water from the compressor tank. Further, the invention comprises a zipper positioned on outer layer of the multilayered Hydrotherapy Blanket, Hydrotherapy Bag, Hydrotherapy Mummy Bag or Hydrotherapy Bed configured to remove air or water bed outer layer for washing or to repair or replace the water bed liner.

In an embodiment of the present disclosure, the AC/DC air compressor is configured to use compressed air to fill the inside of the Hydrotherapy Bed to lift the user off a hard surface, the compressed air is used to wrap the Hydrotherapy Blanket, Hydrotherapy Bag and Hydrotherapy Mummy Bag around the body snugly, the AC/DC air compressor is used to inject compressed air into the hot and cold water to provide a Jacuzzi Hydrotherapy effect. Further, the invention provides one-way air check valve configured to direct the flow of air in one direction.

In an embodiment of the present disclosure, a AC/DC water pump is configured to fill the inside of the Hydrotherapy Bag, Hydrotherapy Mummy Bag, Hydrotherapy Blanket and Hydrotherapy Bed with hot or cold water to provide a Jacuzzi Hydrotherapy massage to the body.

In an embodiment of the present disclosure, the discharge water pump is configured to extract the water from the Hydrotherapy Bed back into the water tank to lower the user back onto a hard surface or back onto the Hydrotherapy air or water mattress built on the outer layer of the Hydrotherapy Blanket, Hydrotherapy Bag, Hydrotherapy Mummy Bag or Hydrotherapy Bed.

In an embodiment of the present disclosure, the plurality of magnetic tubes or the plurality of Hydrotherapy tubes are high-temperature tubing configured to circulate water inside the Hydrotherapy Blanket, Hydrotherapy Bag, Hydrotherapy Mummy Bag or Hydrotherapy Bed.

In an embodiment of the present disclosure, the invention comprises of at least one AC/DC timer and speed controller configured to switch on/off the water pump and to control water flow. Further, the disclosure provides at least one DC fuse or AC electrical circuit breaker to prevent electrical shock.

In an embodiment of the present disclosure, the invention uses at least one AC/DC water heating element, AC/DC hot water thermostat sensor, and an AC/DC hot water thermostat controller configured to heat the water and sense the water temperature and control the water temperature inside the at least one hot water storage tank.

In an embodiment of the present disclosure, the invention comprises an AC/DC water level float to prevent damage to the AC/DC water pump and the other electrical equipment. Further, the invention uses magnetic sensor switches for stopping the flow of water.

The present invention allows hot or cold water to be used to force the ball-bearing magnets through the Magnetic tubes to help with the healing process. The disclosed AC/DC water pump uses a timer or speed control to force the magnets to travel at a certain distance or speed, for better magnetic therapy.

The magnetic sensor relay switches of the invention are installed onto the thermo-fabric to control the AC/DC water pump's flow direction, to move the ball-bearing magnets back and forth. The magnetic sensor relay switches can be adjusted to control the distance of travel for the magnetic ball bearings in a certain area.

The Magnetic tubes will bend or compress, preventing the magnetic ball bearings from traveling inside the magnetic tubes. The present disclosure by applying a long spring inside of the magnetic tubes prevents the Magnetic tubes or Hydrotherapy tubes from collapsing.

There are no known portable devices available that use moving magnets around the body while using high-pressure hot and cold water to help circulate the Red Blood cells, only stationary magnets around the ankles, wrists, skin, or body are available. The disclosed invention creates a moving magnetic field across the skin that produces a magnetic wave, like a sound wave emitted from ultrasound devices. These types of sound wave devices are also like a magnetic field when the magnets are passing across the skin, causing a magnetic wave inside the body.

Severe body injuries resulting in muscle and nerve damage are treated by the therapeutic doctors using an electrical shocking device, called a TENS device (Transcutaneous Electrical Nerve Stimulation) that sends an electrical pulse to the body using electrical pads that makes the muscles twitch and helps the recovery process from nerve and muscle damage.

The disclosed invention uses Silicone or other Waterproof material to waterproof the Silver or Copper Conductive Fabric of Hydrotherapy Blanket, Hydrotherapy Bag, Hydrotherapy Mummy Bag or Hydrotherapy Bed to waterproof the inner layer, the Silver or Copper Conductive Fabric creates an electrical circuit throughout the Hydrotherapy Blanket, Hydrotherapy Bag, Hydrotherapy Mummy Bag or Hydrotherapy Bed when using a TENS Device.

By applying the TENS Device or other types of frequency current to the Copper or Silver Infused Conductive Fabric and sending a current of electricity through the Conductive Fabric that surrounds the inner layer of the Hydrotherapy Bag, Hydrotherapy Mummy Bag, Hydrotherapy Blanket or the top layer of the Hydrotherapy Bed could help in the recovery of injuries.

Hydrotherapy and Dry-hydrotherapy have been used around the World for many years in treating patients with severe injuries to the body. By forcing high-pressure water and air onto the body, studies show it helps improve blood circulation, and reduces swelling of the body tissue, but could also help in developing new muscle tissue.

Human's sweat is something the human body does to remove harmful poisons from the immune system or to cool the body down. By installing magnetic bearing tubes or having the Hydrotherapy tubes sown inside of the Hydrotherapy Blanket, Hydrotherapy Bag, Hydrotherapy Mummy Bag and the Hydrotherapy Bed, and applying hot water inside the Hydrotherapy Bag, Hydrotherapy Mummy Bag, Hydrotherapy Blanket and Hydro-therapeutic bed, generates heat onto the body and causing the body to sweat releasing unwanted harmful particles from the body, but also sweating of the body, helps release some of the fluids that are causing swelling in the joints and allowing faster healing process around the body parts that are injured.

The invention allows a liner made of a magnetic fabric where magnetic bio-ceramic beads are woven into the fabric to create a magnetic fabric. By passing a magnet over the magnetic bio-ceramic beads it helps disperse the magnetic field of the magnetic fabric.

The disclosed Hydrotherapy Mummy Bag, Hydrotherapy Bag and Hydrotherapy Blanket are washable and reusable. The Hydrotherapy tube liner on the Hydrotherapy Bed is removable, washable, and reusable.

The invention allows the pressurized hot and cold water flow to provide a massage using the Hydrotherapy tubes, which the flow of water will go in both directions inside the Hydrotherapy tubes. As the AC/DC water pump will be shut off until the water is released from the Hydrotherapy tubes, but as the water is being released, the water will be forced out of the Hydrotherapy nozzle holes causing a second massage effect upon the body, until the water is completely drained. This method saves on electricity while providing a two-way massage.

The invention allows heat therapy treatment to be applied for 30 minutes to 1 hour, and the nurses or therapists can apply ice water to the Hydrotherapy tubes to reduce the swollen muscles and skin tissue.

The present disclosure allows wheels to be attached to the water tanks with a handle to be able to move the water tanks around.

The present disclosure of the Invention allows the Hydrotherapy Mummy Bag, Hydrotherapy Bag. Hydrotherapy Blanket and Hydrotherapy Bed to use high PSI air along with high-pressurized hot and cold water used for Hydrotherapy during the same Dry-hydrotherapy session, while the user stays dry from a thin water-proof barrier.

The invention uses a waterproof material that is heat and tear-resistant. Hemp fabric is very durable, mold and mildew resistant, and can be waterproofed with a waterproof material like Liquid Rubber or Silicone that is sold in the market. Using an outer layer of thick Hemp fabric, and an inner layer of Hemp fabric that is woven tightly together, by coating both sides of the Hemp fabric with a waterproof coating, which there are many types of rubber, silicone, polymer, waterproof glue, and waterproof adhesives. By adding inside straps sown to the Hemp fabric on both sides of top and bottom fabric of the Hydrotherapy Bed can prevent the water wave motion that waterbeds have, which many water beds uses a foam material inside the waterbeds to prevent water motion. There are also straps going through the Hydrotherapy air mattress or Hydrotherapy water bed that keeps the sides intact and from not bulging out from the sides, which the straps can have hanging eyes on the end of the straps to hang the Hydrotherapy air mattress in the air. Gluing then sowing the waterproof fabric together with a strong sewing thread to keep the waterproof fabric intact and leak-proof. If there needs to be outside stitching, a piece of fabric or leather is sown over the two sown ends of the fabric, to prevent the Hemp fabric from coming apart at the ends of the sown fabric and to prevent any type of leakage.

The invention allows the Hydrotherapy devices to operate on either DC or AC current. Any type of water or oil can be used inside the Hydrotherapy Mummy Bag, Hydrotherapy Bag, Hydrotherapy Blanket or Hydrotherapy Bed. By using Oil this method can increase the pressure of the massage.

The invention allows the portable Hydrotherapy device to be transported in a small vehicle and can be used for Home to Home Dry-hydrotherapy, or used at the gym and in a stadium without the use of AC Current to operate the Hydrotherapy Machine.

The invention allows the Hydrotherapy Mummy Bag, Hydrotherapy Bag, Hydrotherapy Blanket and Hydrotherapy Bed water tubing manifold to use a three-way air Solenoid Valve on the air compressor to shut the air off, until the water pump circulation begins again, and the three-way air Solenoid Valve opens back up and injects high PSI air back into the top or bottom tube manifold to produce a Jacuzzi effect upon the body inside the Hydrotherapy Bag, Hydrotherapy Mummy Bag, Hydrotherapy Bed and Hydrotherapy Blanket.

The invention facilitates the use of hot or cold water at the same time, by having two separate water tanks, to hold hot water and cold water, and by using Solenoid Valves or Motorized Valves, the water pump can be switched to pump either hot or cold water in both directions inside of the Hydrotherapy Bag, Hydrotherapy Mummy Bag, Hydrotherapy Blanket and Hydrotherapy Bed. Further, two water pumps can be used, one for the cold water and one for the hot water, but the water pumps that are needed in Hydrotherapy beds for hospitals and therapy offices need expensive submersible pumps for constant high PSI, so it is cheaper to use three-way Solenoid Valves or Motorized Valves. Add larger pumps to increase PSI and volume of water and add more three-way Solenoid Valves or Motorized Valves to operate more Hydrotherapy Mummy Bags, Hydrotherapy Bags, Hydrotherapy Blankets and Hydrotherapy Beds at the same time to save cost.

The invention provides Ceramic Silicon Nitride ball bearings to be used inside the Hydrotherapy tubes, to simulate a massage like a massage device with roller beads. By forcing high-pressure water inside the Hydrotherapy tubes force the ceramic Silicon Nitride ball bearings to travel up or down inside the Hydrotherapy tubes. The ceramic Silicon Nitride ball bearings have a very smooth surface to glide inside the Hydrotherapy tubes. By using compressed air or water inside the Hydrotherapy Mummy Bag, Hydrotherapy Bag, and Hydrotherapy Blanket to tighten around the body with pressure, increases the massage pressure while using the Ceramic Silicon Nitride ball bearings.

The disclosed Hydrotherapy Mummy Bag, Hydrotherapy Bag and Hydrotherapy Blanket are designed to wrap and cover the patient's body parts to massage the body while sitting in a chair, laying on a massage table or sleeping on a bed.

Since, people who are bedridden or are in a coma, develop bed sores. These types of patients need special therapy each day, which takes the human hands to massage a body, electric shock therapy, or a Jacuzzi to do these types of therapy. People in a Coma cannot be transported and placed into a Jacuzzi without having to use special equipment. The present invention allows the body to be lifted from a hard surface and to lay on top of compressed water or compressed air, and have the ability for comfort to adjust the softness or hardness of the Hydrotherapy Bed, along with adjusting the temperature of the water. The invention allows the body to be bathed on the Hydrotherapy Bed since the removable inner sleeve fabric is made of waterproof fabric or Neoprene. Once the patient is bathed, replace the removable inner sleeve fabric and wash the inner sleeve fabric in the washer. Turn back on the Hydrotherapy Bed to warm the patient back up with hot water.

The claimed invention covers the patient from head to toe inside of a Hydrotherapy Mummy Bag and have their body massaged 24/7, providing better blood circulation throughout the body.

The outer layer of the Hydrotherapy Mummy Bag, Hydrotherapy Bag and Hydrotherapy Blanket contains air or water that can be pressurized around the body parts that need to be massaged. In the inner inside of the Hydrotherapy Mummy Bag, Hydrotherapy Bag, Hydrotherapy Blanket and Hydrotherapy Bed have Hydrotherapy flexible tubes with holes going from end to end of the Hydrotherapy Mummy Bag, Hydrotherapy Bag, Hydrotherapy Blanket and Hydrotherapy Bed. When applying pressurized water into the flexible tubes causes the flexible tubes to expand and generate a water wave to travel up or down all over the skin, muscles, and body to simulate a Jacuzzi effect, by adding constant pressure on the Hydrotherapy Mummy Bag, Hydrotherapy Bag and Hydrotherapy Blanket, help the swelling of the body parts to decrease, but also increases the massage pressure on the Hydrotherapy Mummy Bag, Hydrotherapy Bag and Hydrotherapy Blanket. By forcing high-pressure air and high-pressure hot or cold water into the Hydrotherapy water tubes also massage the injured body parts and helps circulate the body muscles and skin tissue around, to help increase blood circulation.

The disclosed invention is capable of using one or more water pumps, using two-three-way Solenoid Valves or two-three-way Motorized Valves or using a reversible water pump, which provides means for the Hydrotherapy Blanket, the Hydrotherapy Mummy Bag, Hydrotherapy Bag or the Hydrotherapy Bed to be able to circulate compressed hot or cold water back and forth inside the Hydrotherapy tubes. The claimed invention can force the new blood into the injured body parts while forcing the old Blood out of the injured body parts, by using a compressed Hydrotherapy Mummy Bag wrapped around the body, and using a high-pressure water pump to force the hot or cold water to start from the toes as the water flow flows up to the head, then have the compressed hot or cold water flow from the head and back down to the toes, will provide a Hydrotherapy massage as if a patient would have their entire body laying inside a Hydrotherapy Jacuzzi tub, while the user's body stays dry.

The nurse or therapist can use hot water in the first session for 30 minutes to increase the blood flow inside the body and relax and massage the muscles with the Hydrotherapy tubes. After the thirty minutes therapy session, use the cold water for thirty minutes to decrease the swelling of the body parts, while decreasing the blood flow, meanwhile using the Hydrotherapy tubes to massage the body with cold water. In case the patient gets cold chills, simply turn back on the hot water circulation until the patient warms back up, and stops the therapy session.

Pure Oxygen therapy has been used in hospitals and therapy offices to cure wounds using a Hyperbaric Chamber. The Invention allows a AC 120-220 to DC Voltage Converter to supply DC volts to an Anode and Cathode inside Chambers with separator tubes located in the Hydrogen/Oxygen Chambers. This allows pure Oxygen to be generated from H₂O and the Oxygen pumped inside of the compressed Hydrotherapy Bag, Hydrotherapy Blanket or Hydrotherapy Mummy Bag will produce a Hyperbaric Chamber effect. An electrolyte is used to create more Oxygen for the Hydrotherapy Bag, Hydrotherapy Mummy Bag, Hydrotherapy Blanket and Hydrotherapy Bed. By having air tubes going into the Hydrotherapy Bag, Hydrotherapy Mummy Bag or Hydrotherapy Blanket that is compressed around the body, will provide pure Oxygen being injected into the Hydrotherapy Mummy Bag, Hydrotherapy Bag, Hydrotherapy Blanket and Hydrotherapy Bed.

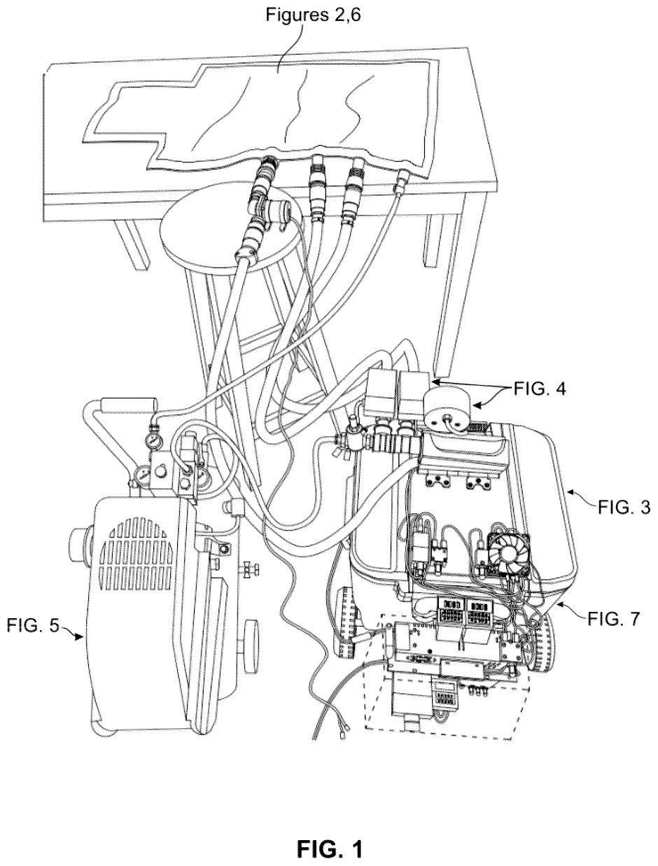

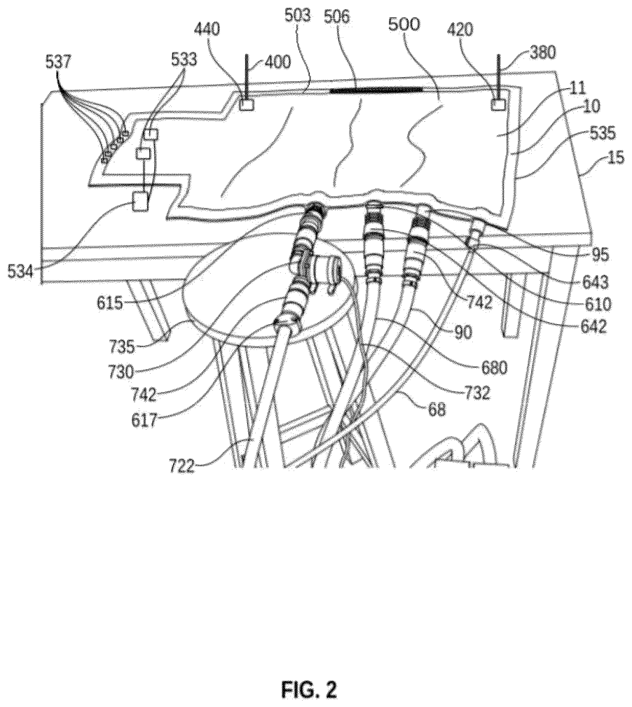

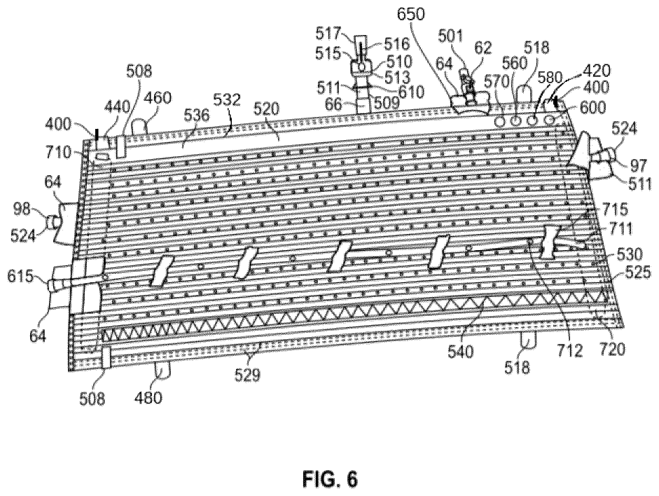

The present invention provides schematics inside view of the Hydrotherapy Mummy Bag, Hydrotherapy Bag, Hydrotherapy Bed or Hydrotherapy Blanket, an air compressor, and the Hydrotherapy Machine for Dry-hydrotherapy body massage as shown in FIG. 1, which also FIG. 1 is configured with FIGS. 2, 3, 4, 5, 6, and 7 which FIG. 2 comprises a Hydrotherapy Mummy Bag, Hydrotherapy Bag or Hydrotherapy Blanket 10 that air is used to compress the Hydrotherapy Mummy Bag, Hydrotherapy Bag or Hydrotherapy Blanket 10 in FIG. 2 snugly around the Body Part while hot or cold water is injected into the Hydrotherapy tubes 525 in FIG. 6 that simulates effects of a Jacuzzi, keeping the Body dry while the Hydrotherapy Mummy Bag, Hydrotherapy Blanket 10 or Hydrotherapy Bed 11 in FIG. 2 can lay on the floor, on top of a massage table or bed 15 in FIG. 2.

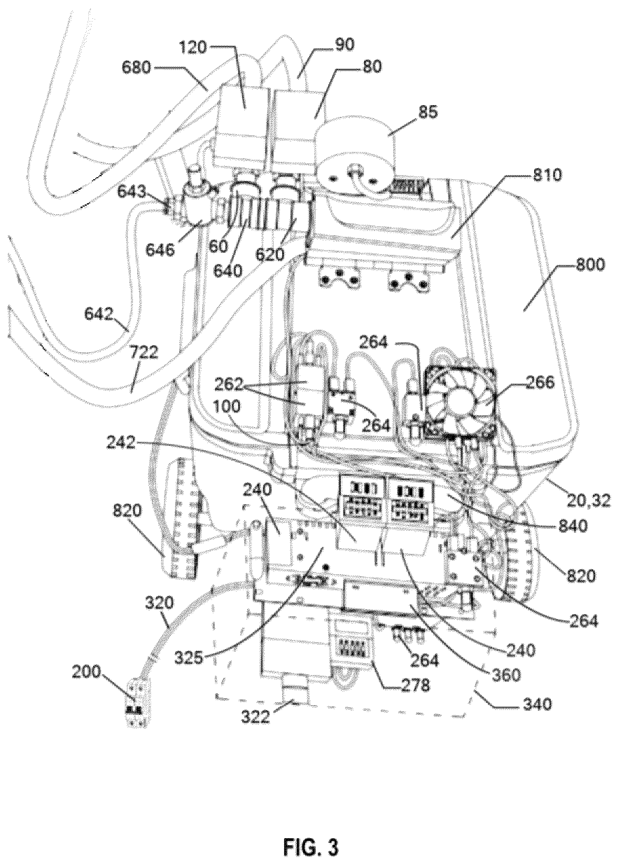

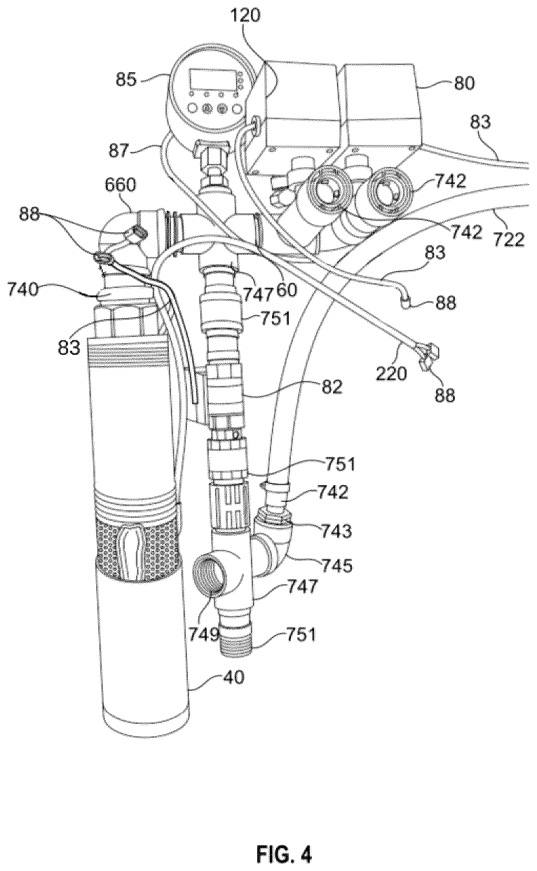

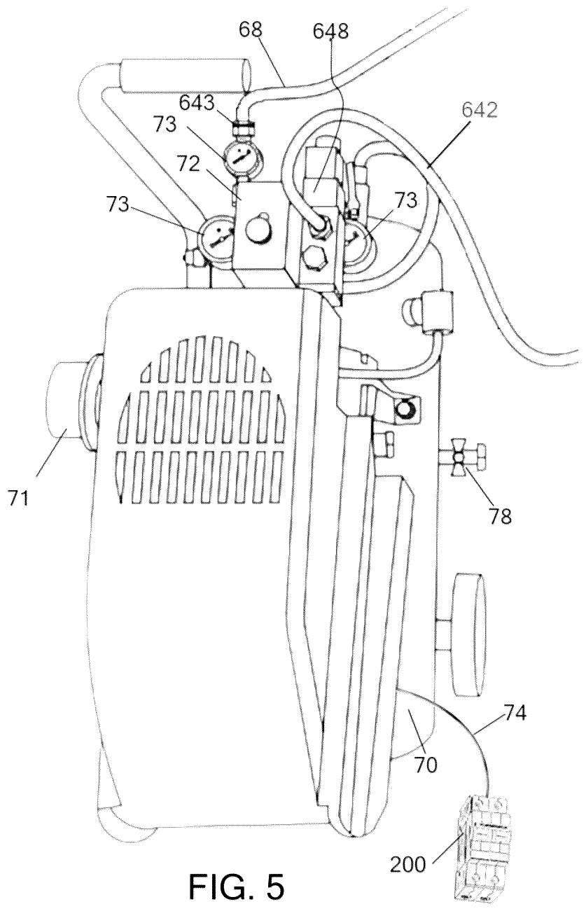

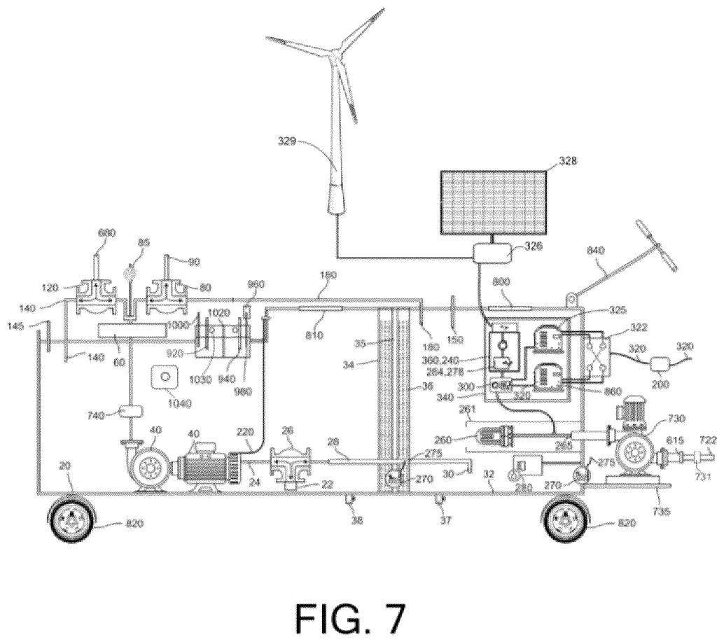

The water storage tanks 20 and 32 in FIGS. 3 and 7 that can store hot and cold water to circulate in the Hydrotherapy Mummy Bag, Hydrotherapy Blanket or Hydrotherapy Bag 10 or the Hydrotherapy Bed 11 in FIG. 2, an inlet water line 22 in FIG. 7 going to the AC/DC 3-Way Solenoid Valve or AC/DC three-way Motorized Valve 26 in FIG. 7 which is attached to the inlet line 24 in FIG. 7 going to the AC/DC hot or cold water pump 40 in Figures and 7 that uses electrical wires 220 in FIGS. 4 and 7 to supply electricity to power the water pump 40 in FIGS. 4 and 7, for circulating the hot or cold water in the Hydrotherapy Mummy Bag, Hydrotherapy Bag, Hydrotherapy Blanket 10 or the Hydrotherapy Bed 11 in FIG. 2, an AC/DC three-way Solenoid Valve or 3-way Motorized Valve 26 in FIG. 7 that allows cold water from tank 20 or hot water tank 32 in FIGS. 3 and 7 to be sucked from the water pump 40 in FIGS. 4 and 7 and the cold or hot water pumped into the Hydrotherapy Mummy Bag, Hydrotherapy Bag, Hydrotherapy Blanket 10 or the Hydrotherapy Bed 11 in FIG. 2, the three-way valve 26 in FIG. 7 is controlled by a timer switch 240 in FIGS. 3 and 7, the insulated hot water inlet line 28 in FIG. 7 keeps the water insulated as it travels to a 3-Way Valve 26 in FIG. 7 to the pump 40 in FIG. 7 from the hot water tank 32 in FIG. 7 into the Hydrotherapy Mummy Bag, Hydrotherapy Bag, Hydrotherapy Blanket 10 or the Hydrotherapy Bed 11 in FIG. 2, a hot water inlet line 30 in FIG. 7 is connected to the 3-Way valve 26 in FIG. 7 that supplies the hot water from the hot water storage tank 32 in FIGS. 3 and 7, the water can be heated with AC/DC water heater elements 260 in FIG. 7, which a water drain tube 261 in FIG. 7 is mounted over the water heating element 260 in FIG. 7, so that the hot water that is drained from the Hydrotherapy Mummy Bag, Hydrotherapy Bag, Hydrotherapy Blanket 10 or Hydrotherapy Bed 11 in FIG. 2, the hot water can be reheated over the water heating element 260 in FIG. 7, the hot water tank separation wall 34 in FIG. 7 covers the insulation 35 in FIG. 7, the cold water tank separation wall 36 in FIG. 7 covers the other side of the insulation 35 in FIG. 7, which is used to insulate the cold water tank 20 in FIG. 7 from the hot water tank 32 in FIG. 7, to keep the temperature of the water the same in each tank, to save on electricity. To save more energy, the hot and cold water tanks' walls can be insulated too, but the Doubled wall water tanks with insulation are not shown in the drawings, because many portable Cold and Hot water tanks with stainless steel walls are built this way with insulation between the stainless steel walls to insulate the water from the atmosphere's temperature. FIG. 3 is a 42 Qt. Portable Plastic Hydrotherapy Machine with Double plastic walls for insulation. Drain valves 37 and 38 in FIG. 7 are used to drain the water from the two water tanks 20 and 32 in FIG. 7 if needed. FIGS. 4 and 7 further shows AC/DC water pump 40 in FIGS. 4 and 7 to circulate the water inside of the Hydrotherapy Mummy Bag, Hydrotherapy Bag or Hydrotherapy Blanket 10, or the Hydrotherapy Bed 11 in FIG. 2, a water pump manifold 640 in FIG. 3 is used to divide the water to circulate the water inside both sides of the Hydrotherapy Mummy Bag, Hydrotherapy Bag, Hydrotherapy Blanket 10 or Hydrotherapy Bed 11 in FIG. 2, an air or water on/off valve adapter 62 in FIG. 6 to the outer liner 500 in FIG. 2, in which air is used to seal the Hydrotherapy Mummy Bag, Hydrotherapy Bag or Hydrotherapy Blanket 10 in FIG. 2 tightly around the body part that needs to be massaged, the air inlet tube seal 64 in FIG. 6 is what seals around the air or water on/off valve adapter 62 in FIG. 6, then a seal clamp 610 in FIGS. 2 and 6 are used to prevent it from leaking air, and an airline quick disconnect 66 in FIG. 6 for an air compressor hose 68 in FIGS. 2 and 5, the air compressor hose 68 in FIGS. 2 and 5 that attaches to the Hydrotherapy Mummy Bag, Hydrotherapy Blanket or Hydrotherapy Bag 10 or the Hydrotherapy Bed 11 in FIG. 2, the AC/DC air compressor with air tank 70 in FIG. 5, using an air filter 71 in FIG. 5 that filters the air before being compressed from the AC/DC Air compressor 70 in FIG. 5 compresses filtered air is injected into the Hydrotherapy Mummy Bag, Hydrotherapy Blanket, Hydrotherapy Bag 10 or the Hydrotherapy Bed 11 in FIG. 2, the air compressor High/Low PSI-On/Off Switch 72 in FIG. 5 so that the Hydrotherapy Blanket, Hydrotherapy Mummy Bag, Hydrotherapy Bag 10 or the Hydrotherapy Bed 11 in FIG. 2 can be adjusted according to the need, as Regulators 73 in FIG. 5 is used to control the PSI.

The FIG. 5 further shows AC/DC wires 74 to operate the air compressor 70 in FIG. 5, an air compressor water drain valve 78 in FIG. 5 to drain the water from the compressor tank 70 in FIG. 5, a Two/Three-Way fluid Solenoid Valve or a 2 or 3-Way Electrical Motorized On-Off Valve 120 in FIGS. 3, 4 and 7 that allows water to be pumped into the Hydrotherapy tubes 525 in FIG. 6, as the other fluid 3-Way Solenoid Valve or a 3-Way Electrical Motorized Valve 120 in FIGS. 3, 4 and 7, allows water to be injected on the other end of Hydrotherapy tubes 525 in FIG. 6 which provides a Hydrotherapy massage in both directions inside of the Hydrotherapy Mummy Bag, Hydrotherapy Bag, Hydrotherapy Blanket 10 and Hydrotherapy Bed 11 in FIG. 2, the water is returned back into the water storage tanks 20 or 32 in FIGS. 3 and 7 if a 3-Way Valves 120 and 80 in FIGS. 3, 4 and 7 are used. In FIG. 7 the High-Pressure/Low-Pressure Fluid Solenoid Valves or Motorized Valves 80 and 120 in FIGS. 3, 4 and 7 that inflates and circulates the hot or cold water throughout the Hydrotherapy tubes 525 in FIG. 6 that are located inside the Hydrotherapy Mummy Bag, Hydrotherapy Bag, Hydrotherapy Blanket 10 or Hydrotherapy Bed 11 in FIG. 2, the Inlet Water Line Hose 90 in FIGS. 2, 3, and 7 and the inlet water line 680 in FIGS. 2, 3, and 7 goes to the Hydrotherapy Mummy Bag, Hydrotherapy Bag, Hydrotherapy Blanket 10 or Hydrotherapy Bed 11 in FIG. 2 to inject water and air into the Hydrotherapy Mummy Bag, Hydrotherapy Bag, Hydrotherapy Blanket 10 or Hydrotherapy Bed 11 in FIG. 2, the Inlet Water Tube Connection 97 in FIG. 6 to allow water to enter into the Hydrotherapy Mummy Bag, Hydrotherapy Bag, Hydrotherapy Blanket 10 or Hydrotherapy Bed 11 in FIG. 2, the Water Line Inlet Tube Seal 95 in FIG. 2 that seals around the Inlet Water Tube or hose connection 97 in FIG. 6 to prevent Water or Air from Leaking out of the Hydrotherapy Bag. Hydrotherapy Mummy Bag, Hydrotherapy Blanket 10 and Hydrotherapy Bed 11 in FIG. 2 as the opposite Inlet water tube/hose connection 98 in FIG. 6 forces water into the opposite direction into the Hydrotherapy Mummy Bag, Hydrotherapy Bag, Hydrotherapy Blanket 10 or Hydrotherapy Bed 11 in FIG. 2.

The AC/DC Timer Wires 100 in FIG. 3 going to the Fluid Solenoid Valve or 2 or 3-Way Electrical Motorized Valves 80 and 120 in FIGS. 3, 4 and 7 that controls the flow of the Water to the Hydrotherapy Mummy Bag, Hydrotherapy Bag, Hydrotherapy Blanket 10 or Hydrotherapy Bed 11 in FIG. 2, the 2 or 3-Way Fluid Solenoid Valve or 2 or 3-Way Motorized Valve 120 in FIGS. 3, 4 and 7 controls the flow of the water to the opposite end of the Hydrotherapy Mummy Bag, Hydrotherapy Bag, Hydrotherapy Blanket 10 or Hydrotherapy Bed 11 in FIG. 2 and releases the Water Load back into the Cold Water Storage Tank 20 in FIG. 7, a Cold Water Dump Line 140 in FIG. 7 connected to the 3-Way Valve 120 in FIG. 7 to release the Cold Water out of the Hydrotherapy Tubes 525 in FIG. 6 into the Cold Water Storage Tank 20 in FIG. 7, and a Cold Water Dump Line 145 in FIG. 7 to release the Hot Water out of the Hydrotherapy Tubes 525 in FIG. 6 into the Hot Water Storage Tank 32 in FIG. 7, the Hot Water Dump Line 150 in FIG. 7 releases the hot water out of the Hydrotherapy tubes 525 in FIG. 6 in the Hot Water Storage Tank 32 in FIGS. 3 and 7, the 2-Way Fluid Solenoid Valve or a 2-Way Electrical Motorized On-Off Valve 82 in FIG. 4 helps with draining the Hydrotherapy Mummy Bag, Hydrotherapy Bag, Hydrotherapy Blanket 10 or Hydrotherapy Bed 11 in FIG. 2, but 82 in FIG. 4 also helps to decrease the flow of water into the Hydrotherapy Mummy Bag, Hydrotherapy Bag, Hydrotherapy Blanket 10 or Hydrotherapy Bed 11 by slowly closing the water valve while it is opened, as the electrical wires 83 in FIG. 4 provide the electricity to operate the Fluid Solenoid Valve or a 2-Way Electrical Motorized On-Off Valve 82 in FIG. 4. Note that the 3-Way valves 120 and 80 in FIGS. 3, 4 and 7 can be used to drain the water from the Hydrotherapy Mummy Bag, Hydrotherapy Bag, Hydrotherapy Blanket 10 or Hydrotherapy Bed 11 or Hydro-Therapeutic bed 11 in FIG. 2 faster, if a positive displacement water pump is being used.

The High pressure/low pressure Solenoid Valve or manual pressure control valve 85 in FIGS. 3, 4 and 7 controls the water PSI going to the Hydrotherapy Mummy Bag, Hydrotherapy Bag, Hydrotherapy Blanket 10 or Hydrotherapy Bed 11 in FIG. 2, while the High pressure/low pressure Solenoid Valve electrical wires 87 in FIG. 4 that uses wire terminals 88 in FIG. 4 to connect to the Relay timer switch 240 in FIGS. 3 and 7, the AC/DC Timer switch 240 in FIGS. 3 and 7 controls the time to open and close the Solenoid Valves or Motorized Valves 120, 80 in FIGS. 3, 4 and 7 and the 2-Way Valve 82 in FIG. 4.

The AC/DC Timer 240 wires 100 in FIG. 3 that are connected to the Solenoid Valves or Motorized Valves 120, and 80 in FIGS. 3, 4 and 7, the Water Dump Line 140 in FIG. 7 to release the Cold Water out of the Hydrotherapy Tubes 525 in FIG. 6 in the Cold Water Storage Tank 20 in FIG. 7 and the hot water three-way valve 120 water dump line 180 in FIG. 7 returns the hot water into the hot water tank 32 in FIG. 7.

The DC Fuse/AC Breaker 200 in FIGS. 3, 5 and 7 is used on the Hydrotherapy device to prevent an Electrical Shortage to the Equipment or Electrical Shock to the Body, the AC/DC Wires 220 in FIGS. 4 and 7 for the Water Pump 40 in FIGS. 4 and 7 is connected to an AC/DC Water Pump Timer and Speed Control Switch 242 in FIG. 3 that controls the Speed of the Water Pump 40 in FIGS. 4 and 7 and controls the On/Off time for the Water Pump 40 in FIGS. 4 and 7, the Water Pump 40 in FIGS. 4 and 7 will be turned off for a few seconds to allow the Hydrotherapy Tubes 525 in FIG. 6 to release the Water and collapse the Hydrotherapy tubes 525 in FIG. 6. When Timer 242 in FIG. 3 activates the Water Pump 40 in FIGS. 4 and 7 causes the Hydrotherapy Tubes 525 in FIG. 6 to expand from the High-Pressure Water and the Timer 242 in FIG. 3 repeats until the therapy is completed.

The hot water timer 278 in FIGS. 3 and 7 turns off and on the water heater element 260 in FIG. 7, and a AC 4-way On/off power switch 322 in FIGS. 3 and 7 is attached to the Electrical box 340 in FIGS. 3 and 7 to control the On/Off on the AC to DC voltage converter 325 in FIGS. 3 and 7 and AC to DC voltage converter 860 in FIG. 7 which are used to convert AC electricity to DC electricity to operate the water pump 40 in FIGS. 4 and 7 and other 12V electrical parts, so that 12 Volts DC can be used to prevent any type of deadly electrical shock around water, the Hydrotherapy Machine in FIGS. 3 and 7 is portable and can operate off of DC Batteries 326 in FIG. 7, operate off of DC Solar Panels 328 in FIG. 7 or operate using a portable Wind Turbine Generator 329 in FIG. 7, the Electrical box 340 in FIGS. 3 and 7 stores the AC to DC Converters, but also can store more Relay Switches if needed, the Two-Three-Four-Way On/Off Remote Control Relay Switch 360 in FIGS. 3 and 7 controls Timer Relay Switches 240 and the Timer Relay Switch 240 in FIGS. 3 and 7 controls the Solenoid Valves or Motorized Valves to Open and Close by a remote Control or controlled by other types of Timer Relay Switches, the Two-Three-Four-Way On/Off Remote Control Relay Switch 360 in FIGS. 3 and 7 is used to turn off the water pump 40 in FIGS. 4 and 7 and the heating element 260 in FIG. 7, the AC/DC Wires 380 in FIG. 2 going from the Magnetic Switches 420 and 440 in FIGS. 2 and 6 are connected to the Water Pump Timer 242 in FIG. 3 to turn off the Water Pump 40 in FIGS. 4 and 7 when the Water has filled up the Magnet Ball Bearing Tubes 520 in FIG. 6, the AC/DC Wires 400 in FIG. 2 and FIG. 6 going from Magnetic Switch 420 in FIGS. 2 and 6 from the Relay Switch 360 in FIGS. 3 and 7, the Top Magnetic Switch 420 in FIG. 2 and FIG. 6 that controls the Water Flow flowing through the Magnetic tubes 520 in FIG. 6 in one direction to give a Full Body Massage from the Head to the Toes.

FIG. 7 further shows the Heater Element Adapter 265 that holds in place the AC/DC Heater Element 260 in FIG. 7, the AC/DC relays switches 262 in FIG. 3 control the on and off for Heater Elements 260 in FIG. 7, if the AC/DC relay switches 262 in FIG. 3 gets hot, a AC/DC fan 266 in FIG. 3 is used to lower the temperature, as a AC/DC Timer 278 in FIGS. 3 and 7 is used to set the time for the heater element thermostat controller 300 in FIG. 7 to be turned on and off, the hot and cold water tanks' Water Level Floats 270 in FIG. 7 to turn off water pump 40 in FIGS. 4 and 7 when no Water is detected. Further, the AC/DC wires 275 in FIG. 7 going to and from the water level floats 270 in FIG. 7 to the Relay Switch 360 in FIGS. 3 and 7 to shut down the Hydrotherapy machines FIGS. 3 and 7 from operation, the AC/DC Hot Water Thermostat Sensor 280 in FIG. 7 that senses the Temperature of the water, the AC/DC Hot Water Thermostat Controller 300 in FIG. 7 is what controls the Temperature of the Water inside the Hot Water Tank 32 in FIG. 7, the AC/DC Main Electrical Wires 320 in FIGS. 3 and 7 that connects to the Thermostat Controller 300 in FIG. 7, the Electrical Storage Box 340 in FIGS. 3 and 7 that protects the DC Breakers 264 in FIGS. 3 and 7, the Two-Three-Four-Way On/Off remote control relay switch 360 in FIGS. 3 and 7 and relay switch timer 240 in FIGS. 3 and 7, using the remote controller relay switches 360 in FIGS. 3 and 7 the user or the Therapist at a distance can operate FIG. 3 Hydrotherapy Machine's water pump 40 in FIGS. 4 and 7 and heater controller timer 278 in FIGS. 3 and 7.

Magnetic sensor relay switch 440 in FIG. 2 and FIG. 6 that controls the Water Flow flowing through the Hydrotherapy tubes 525 in FIG. 6 in the other direction from the Toes to the Head, the Velcro Hooks 460 and Velcro Fabric 480 in FIG. 6 used to strap down the Hydrotherapy Bag, Hydrotherapy Mummy Bag and Hydrotherapy Blanket 10 in FIG. 2 around the Body Parts snugly, the Outer Layer 500 in FIG. 2 of the Hydrotherapy Mummy Bag, Hydrotherapy Bag or Hydrotherapy Blanket 10 in FIG. 2 that contain High-PSI Air or Water to compress the Hydrotherapy Mummy Bag, Hydrotherapy Bag or Hydrotherapy Blanket 10 around the body tightly, a Thermo-Fabric like Neoprene Fabric helps the Water keep the Temperature that is needed for Therapy, leather can also be used on the outer layer 500 in FIG. 2 on the Hydrotherapy Mummy Bag, Hydrotherapy Blanket, Hydrotherapy Bag 10 or Hydrotherapy Bed 11 in FIG. 2 to help insulate, but also provide a tougher layer and longer life to the Hydrotherapy Blanket, Hydrotherapy Bag, Hydrotherapy Mummy Bag 10 or Hydrotherapy Bed 11 in FIG. 2, the Outer Layer 500 in FIG. 2 can also be made from either; Leather, Vinyl, Fabric or Waterproof Fabric that is made from Nylon, Cotton or Hemp Fabric

Air or Hot/Cold Water 501 in FIG. 6 that is used between the Outer Layer Fabric 500 in FIG. 2 and the Hydrotherapy tubes 525 in FIG. 6 of the Waterproof Fabric, an Inner Air or Water Bed Liner 500 in FIG. 2 that can either store Air or Water, the Water Bed Liner 503 in FIG. 2 is removable and washable and can be made of Vinyl, waterproof Cotton or Nylon, or preferable Waterproof Hemp Fabric that will last longer than the other Materials used to make Air Mattresses or Water Beds.

The figure further shows the Zipper 506 in FIG. 2 to allow the Liner 503 in FIG. 2 of the Hydrotherapy Bed 11 in FIG. 2 to be removed and washed, Hydrotherapy Water Bed or Air Mattress Outer Liner 500 in FIG. 2 that supports the Air or Water Bed Liner 503 in FIG. 2, the Support Straps 508 in FIG. 6 to prevent the Water Bed or Air Mattress Outer Liner 500 in FIG. 2 from sloshing around, like how the Water Beds that use no Foam.

The Air or the Water Inlet Fittings 509 in FIG. 6 that is attached to the Ball Check Valve 510 in FIG. 6, and sealed by the Water Inlet Fitting Seal 511 in FIG. 6, the seal clamp 610 in FIGS. 2 and 6 is used to prevent the water from leaking out, the Ball Check Valve 515 in FIG. 6 that is used to prevent the Air or Water from being released as the Hose or Air Line is released, the Water Inlet Fitting Seal 511 in FIG. 6 goes around the Water Inlet Fitting 509 in FIG. 6 to prevent from Leaking, the Quick connect and disconnect hose connector adapter 615 in FIGS. 2, 6 and 7 can be used to connect the hoses or a hose clamp 617 in FIG. 2 can be used to attach the hoses 680 in FIGS. 2, 3, and 7 with the Fluid Solenoid Valve or a 2 or 3 Way Electrical Motorized On-Off Valve 80 in FIGS. 3, 4 and 7.

The Check Valve Ball Stopper 513 in FIG. 6 to prevent the Ball Check Valve 515 in FIG. 6 from blocking the flow of Air or Water, the Check Valve Ball 515 in FIG. 6 to prevent the Air or Water from leaving the Hydrotherapy Air Mattress or Hydrotherapy Water bed 11 in FIG. 2, the Check Valve Ball Pin 516 in FIG. 6 releases the Check Valve Ball 515 in FIG. 6 and allow the Air or Water from escaping from the Outer Layer 500 in FIG. 2 for the Hydrotherapy Air Mattress or Hydrotherapy Water Bed 11 in FIG. 2, the Hose or Air Adapter 517 in FIG. 6 with Check Valve Ball Pin 516 in FIG. 6 is attached inside hose connection adapter 517 in FIG. 6, to allow Air Hose or a Water Hose to Connect to the Ball Check Valve 510 in FIG. 6, the Eye Loop 518 in FIG. 6 to secure Bed Sheets or to Hang the Hydrotherapy Air Mattress, the Magnetic Ball Bearing Tubes 520 in FIG. 6 that is used inside the Magnetic tubes 520 in FIG. 6 and the Hydrotherapy Tubes 525 in FIG. 6 that allow Hot or Cold Water to flow through the Hydrotherapy Mummy Bag, Hydrotherapy Bag, Hydrotherapy Blanket 10 or Hydrotherapy Bed 11 in FIG. 2.

The Hydrotherapy Nozzle Holes 530 in FIG. 6 allows High Pressured Air and Water to travel out of the Hydrotherapy Tubes 525 in FIG. 6 to massage the Body like a Hydrotherapy does, without having the Water touching the Skin, the Hot and Cold Water Flow direction 524 in FIG. 6, which the flow of Water will flow both directions inside the Hydrotherapy Tubes 525 in FIG. 6 using the hose connection adapters 97 and 98 in FIG. 6, the Water Pump will be shut off until the water is released from the Hydrotherapy Tubes 525 in FIG. 6, but as the Water is being released the Water will be forced out of the Hydrotherapy Nozzle Holes 523 in FIG. 6 causing a Second Massage Effect upon the Body, until the Water is completely drained. This Method saves on Electricity while providing a Two Way Massage.

FIG. 6 further shows the Sew Stitching 529 that is used to form the Hydrotherapy Tubes 525 in FIG. 6, the Sew Stitching 529 in FIG. 6 also is used to Stitch and hold together the Waterproof layers that are used to fabricate the Hydrotherapy Mummy Bag, Hydrotherapy Bag and Hydrotherapy Blanket 10 or the Hydrotherapy Bed 11 in FIG. 2, the Hydrotherapy Tube Nozzle Holes 530 in FIG. 6 that are in the Hydrotherapy Tubes 525 in FIG. 6 and when high pressured air and Water is forced out of the Hydrotherapy Tubes 525 in FIG. 6 through the Hydrotherapy Tube Nozzle Holes 530 in FIG. 6, causes a High-Pressure Air and Water Spray to be applied to the Waterproof Inner Layer 535 in FIG. 2, while also causing a Hydrotherapy Massage effect onto the Body, like how a Hydrotherapy Nozzle in a Jacuzzi works.

In FIG. 6 it further shows the Silicone or other Waterproof material 532 used to Waterproof the Copper or Silver Infused Conductive Fabric 536 in FIG. 6, the Copper or Silver Infused Conductive Fabric 536 used to enhance the range of the TENS Device (Transcutaneous Electrical Nerve Stimulation), the TENS Device's Positive and Negative Wire Connections 533 in FIG. 2 that are connected to Copper or Silver Infused Conductive Fabric, the TENS Device 534 (Transcutaneous Electrical Nerve Stimulation) in FIG. 2 that is used to stimulate the Muscles and Nerves, the Thin Waterproof Inner Liner 535 in FIG. 2 of the Hydrotherapy Mummy Bag, Hydrotherapy Bag and Hydrotherapy Blanket 10 and Hydrotherapy Bed 11 in FIG. 2, which allows the skin to stay dry, but allows the body to be massaged by using the Hydrotherapy Holes 530 in FIG. 6 that sprays hot or cold water onto the inner layer waterproof fabric of the Hydrotherapy Mummy Bag, Hydrotherapy Bag, Hydrotherapy Blanket 10 or the Hydrotherapy Bed 11 in FIG. 2, the Finger/Toe Straps 537 in FIG. 2 allow the Fingers or Toes to be strapped in, and as the Hydrotherapy Bag expands using either Air or Water or Both together, which will cause the Hydrotherapy Bag to expand out the Fingers or toes. Using this same Method can be used or Rubber Bands can be attached to the Hydrotherapy Bag to Collapse the Fingers and Hands. Further, the Springs 540 in FIG. 6 that goes inside the Magnetic tubes 520 in FIG. 6 or Hydrotherapy tubes 525 in FIG. 6, to prevent the Hydrotherapy tubes from collapsing when being used with Water, the Magnetic Ball Bearings 600 in FIG. 6 are inside of the Magnetic tubes 520 in FIG. 6, the Magnets are used to provide a Magnetic Field and the Magnetic Bearings 600 in FIG. 6 roll inside the Magnetic Tubes 520 in FIG. 6.

The Ceramic Silicon Nitride Ball Bearings 570 in FIG. 6 that are used inside of the Magnetic Tubes 520 in FIG. 6 provides a massage, and the Plastic Ball Bearing Separator 580 in FIG. 6 is used to Separate the Magnetic Field and prevent Magnetic Ball Bearings 600 in FIG. 6 and the extra Magnetic Ball Bearings 560 in FIG. 6 from sticking together, when two or more Magnetic Bearings 560 in FIG. 6 are used to increase the Magnetic Field inside the Magnetic Tubes 520 in FIG. 6, the Manifold Tube Inlets 620 in FIG. 3 allows the Water to be injected into the Magnetic Tubes 520 in FIG. 6 or the Hydrotherapy tubes 525 in FIG. 6, the Tubing Manifold fitting 620 in FIG. 3 allows the Water to be injected into the Water Pump Manifold 640 in FIG. 3, the Air Hose 642 in FIGS. 3 and 5 is attached to the airline connections 643 in FIGS. 2, 3 and 5 as an airline connection 643 is attached to a one-way check valve 646 in FIG. 3, as the one-way check valve 646 allows air to travel one way into the Water Pump Manifold 640 in FIG. 3 and prevents water going into the Air Compressor Hose 642 in FIGS. 3 and 5.

The Hydrotherapy Nozzle Holes 530 in FIG. 6 as this Method allows high-pressure Water and Air to be injected out of the Hydrotherapy Tubes 525 in FIG. 6, to Simulate the effects of what the Jacuzzi effects are, the Air Solenoid Valve 648 in FIG. 5 shuts off the Air Flow when water pump 40 in FIGS. 4 and 7 is off, the Air Hose Adapter Seal 650 in FIG. 6 seals around the Air On/Off valve Adapter 62 in FIG. 6 going into the Hydrotherapy Mummy Bag, Hydrotherapy Bag, Hydrotherapy Blanket 10 and Hydrotherapy Bed 11 in FIG. 2 to prevent Water and Air from leaking, the Water Pump Manifold 640 in FIG. 3 is used to direct the water going into the Hydrotherapy Mummy Bag, Hydrotherapy Bag, Hydrotherapy Blanket 10 and Hydrotherapy Bed 11 in FIG. 2, the 90 Degree Tubing Connection 6 in FIG. 4 is connected to the Water Manifold 640 in FIG. 3, the Inlet-Outlet Insulated Water Line 680 in FIGS. 2, 3 and 7 circulates the Hot and Cold Water from the Water Pump 40 in FIGS. 4 and 7.

Note that the water pump 40 in FIGS. 4 and 7 can be either a submersible water pump or a water pump not made to be submersed in water, but needs to be mounted outside the water tanks on water pump mount 375 in FIG. 7, the Top Water Manifold 710 in FIG. 6 and the Bottom Water Manifold 720 in FIG. 6, is to allow Water Flow in both directions inside of the Hydrotherapy tubes 525 in FIG. 6, the outlet hose 711 in FIG. 6 is used to drain the Hydrotherapy Mummy Bag, Hydrotherapy Bag, Hydrotherapy Blanket 10 or Hydrotherapy Bed 11 in FIG. 2 from the outlet water tube holes 712 in FIG. 6 that are used to drain the entire Hydrotherapy Mummy Bag, Hydrotherapy Bag, Hydrotherapy Blanket 10 and Hydrotherapy Bed 11 in FIG. 2, while the outlet water tube 711 in FIG. 6 is tied down by hose straps 715 in FIG. 6, the Outlet drain hose 722 in FIGS. 2, 3, 4 and 7 drains the water back into the water tank 20 in FIG. 3, the AC/DC Water drain pump 730 in FIGS. 2 and 7 is attached to a AC/DC water sensor switch 731 in FIG. 7 which detects if there is a water flow coming out of the Hydrotherapy Mummy Bag, Hydrotherapy Bag, Hydrotherapy Blanket 10 or Hydrotherapy Bed 11 in FIG. 2 and shuts off the water drain pump 730 in FIGS. 2 and 7 when the water is drained from the Hydrotherapy Mummy Bag, Hydrotherapy Bag, Hydrotherapy Blanket 10 or Hydrotherapy Bed 11 in FIG. 2, the AC/DC Water drain pump 730 in FIGS. 2 and 7 helps extracting the water faster out of the Hydrotherapy Mummy Bag, Hydrotherapy Bag, Hydrotherapy Blanket 10 or Hydrotherapy Bed 11 in FIG. 2, but also aids in pumping the water higher than what the elevation of the Hydrotherapy Mummy Bag, Hydrotherapy Bag, Hydrotherapy Blanket 10 or Hydrotherapy Bed 11 in FIG. 2, the AC/DC Water drain pump wires 732 in FIG. 2 are connected to the Water Pump Timer 242 in FIG. 3, the pump stand 735 in FIG. 2 and FIG. 7 can be used to mount the drain pump 730 in FIGS. 2 and 7.

The Hot Water Storage Tank Lid 800 and cold water tank lid 810 in FIGS. 3 and 7 are used to add or to drain the Water. FIGS. 3 and 7 further shows the Cart Wheels 820 are used to move the Storage Water Tank, the Cart Handle 840 in FIGS. 3 and 7 used to move Storage Water Tank in FIGS. 3 and 7, the AC voltage to DC voltage converter 860 in FIG. 7 changes AC 120-220 to DC voltage to supply DC Volts to a Variable AMP DC Voltage Transformer 1040 in FIG. 7 that supplies DC Voltage to the Cathode 920 in FIG. 7 and to the Anode 940 in FIG. 7, the Cathode 920 and the Anode 940 in FIG. 7 are used to create pure Oxygen and Hydrogen Gas from H₂O, the Oxygen is injected inside the Hydrotherapy Mummy Bag, Hydrotherapy Bag, Hydrotherapy Blanket 10 or Hydrotherapy Bed 11 in FIG. 2, as the Oxygen Line 960 in FIG. 7 going from the Oxygen Separator Tube 980 in FIG. 7 which Oxygen Line 960 in FIG. 7 travels inside of the Hydrotherapy Mummy Bag. Hydrotherapy Bag, Hydrotherapy Blanket 10 or Hydrotherapy Bed 11 in FIG. 2 to provide pure Oxygen to the Body, the Oxygen Separator Tube 980 in FIG. 7 that contains the Oxygen that is separated from H₂O, the Hydrogen Gas Separator Tube 1000 in FIG. 7 that contains the Hydrogen Gas is escaped from Hydrogen Gas Separator Tube 1000 in FIG. 7 where the Hydrogen Gas and Oxygen is produced inside the Oxygen/Hydrogen Gas Chambers 1020 in FIG. 7 that holds Water and an Electrolyte to create Oxygen for the Hydrotherapy Mummy Bag, Hydrotherapy Bag, Hydrotherapy Blanket 10 or the Hydrotherapy Bed 11 in FIG. 2, the Hydrogen gas explosion vent hole 1030 in FIG. 7 can be used to prevent the Hydrogen gas from expanding and exploding inside the Oxygen/Hydrogen Gas Chambers 1020 in FIG. 7, the Hydrogen Gas that is created by creating Oxygen from H₂O, the Hydrogen Gas can be used to heat the water for Hydrotherapy, and to save on the cost, by heating water from other means.

The 90 Degree Connection 660 in FIG. 4 is a Water Valve Connection 60 in FIGS. 3, 4 and 7 on the Water Manifold 640 in FIG. 3 to connect the Water Valves 80 and 120 in FIG. 7, the Union 740 in FIGS. 4 and 7 allows the water pump 40 to connect and disconnect from the Water Pump manifold 640 in FIG. 3, the quick disconnect 742 in FIGS. 2 and 4, which is connected to the hose connection adapter 743 in FIG. 4, the quick disconnect 742 in FIGS. 2 and 4 can be used on the Hydrotherapy Mummy Bag, Hydrotherapy Bag, and Hydrotherapy Blanket 10 in FIG. 2, for quicker connects and disconnects for the drain hose 722 in FIGS. 2, 3, 4 and 7 which the drain hose 722 goes to water tank 20 and connected to a 90 degree pipe fitting 745 in FIG. 4, and then connected to a 4-way tubing connection 747 in FIG. 4, which is used for the Water drain outlet port 749 in FIG. 4, by adding pipe fitting nipples 751 in FIG. 4 are used to draw more of a suction towards the bottom of the Water Storage Tanks 20 and 32 in FIGS. 3 and 7, while the drain water from the Hydrotherapy Mummy Bag. Hydrotherapy Bag, Hydrotherapy Blanket 10 and Hydrotherapy Bed 11 in FIG. 2 is being drained or the direction of the water flow can pass over the water heating element 260 in FIG. 7.

The disclosed invention can be used in homes, gyms, therapy offices, and hospitals at an affordable price. Also, it occupies lesser space as compared to the space required by the conventional therapy Jacuzzi, while using 100's of gallons of less water than the conventional Hydrotherapy Jacuzzi.

Below mentioned is the list of reference numerals used in the figures:
10 Hydrotherapy Mummy Bag/Hydrotherapy Bag/Hydrotherapy Blanket
11 Hydrotherapy Mattress/Bed
15 Table or Bed
20 Cold water storage tank
22 Inlet water line for Solenoid Valve or Motorized Valve
24 Inlet cold water line for pump
26 AC/DC 3-Way Solenoid Valve or 3-Way Motorized Valve
28 Insulated hot water inlet line to pump
30 Hot water inlet line to 3-Way valve
32 Hot water storage tank
34 Hot water storage tank separation wall
35 Insulation
36 Cold water storage tank separation wall
37 Hot water tank drain valve
38 Cold water tank drain valve
40 AC/DC hot and cold water pump
60 Water valve connection
62 air or water on/off valve adapter
64 Air inlet tube seal
66 Air line quick disconnect
68 Air Hose to Hydro-Therapeutic Bag
70 AC/DC air compressor with Air Tank
71 Air Compressor Air Filter
72 Air compressor High/Low PSI on/off switch
73 Air Regulators
74 AC/DC wires for air compressor
78 Air Tank Water Drain Valve
80 Fluid Solenoid Valve or a 2 or 3-Way Electrical Motorized On-Off Valve
82 Outlet Fluid Solenoid Valve or a 2-Way Electrical Motorized On-Off Valve
83 Outlet Fluid Valve electrical wires
85 High pressure/low pressure Solenoid Valve or manual pressure control valve
87 High pressure/low pressure Solenoid Valve electrical wires
88 Wire Terminals
90 Inlet water line hose
95 water line inlet tube seal
97 Inlet water/hose connection
98 Opposite Inlet water tube/hose connection
100 AC/DC Timer electrical wires
120 AC/DC Fluid Solenoid Valve or a 2 or 3-Way Electrical Motorized Valve
140 Cold water valve dump line
145 Cold water dump line
150 Hot water dump line
180 Hot water valve dump line
200 DC Fuse/AC breaker
220 AC/DC wires for water pump
240 AC/DC water pump timer relay switch
242 AC/DC Timer and speed control for water pump
260 AC/DC hot water heater element
261 Outlet water drain tube
262 DC Relay Switches
264 DC Breakers
265 Heater element adapter
266 Fan
270 AC/DC water level floats
275 AC/DC water level float wires
278 AC/DC Timer for Hot water heating elements
280 AC/DC hot water thermostat sensor
300 AC/DC hot water thermostat controller
320 AC/DC main electrical wires
322 AC/DC 4-Way On/Off switches
325 AC to DC voltage converters
326 DC Batteries
328 DC Solar Panel
329 Portable Wind Turbine Generator
340 Electrical storage box
360 Two-Three-Four-Way On/Off remote control relay switch
380 AC/DC wires going from magnetic switches
400 AC/DC wires going from magnetic switches
420 Top Magnetic sensor relay switch
440 Bottom Magnetic Sensor Relay switch
460 Velcro hook
480 Velcro Fabric
500 Outer layer for Hydrotherapy Bag, Hydrotherapy Blanket or Hydrotherapy Bed
501 Air/water
503 Inner air or water bed liner
506 Hydrotherapy tube cover zipper
508 Support Straps
509 Air or Water Inlet Fitting
510 Ball Check Valve
511 Water Inlet Fitting Seal
513 Check Valve Ball Stopper
515 Check Valve Ball
516 Check Valve Ball Pin
517 Hose or Air Adapter with Check Valve Ball Pin
518 Tie Down Eye Loops
520 Magnetic Tubes
524 Hot or Cold Water Flow
525 Hydrotherapy tubes
529 Sew Stitching
530 Hydrotherapy Tube Nozzle Holes
532 Silicone or other Waterproof material
533 TENS Device Electrode Positive and Negative Wire Connections
534 TENS Device (Transcutaneous Electrical Nerve Stimulation)
535 Thin Waterproof Inner Liner
536 Copper or Silver Infused Conductive Fabric
537 Finger/Toe Straps
540 Springs
560 Magnetic Ball Bearing
570 Ceramic Silicon Nitride Ball Bearings
580 Plastic Ball Bearing Separator
600 Magnetic Ball Bearings
610 Seal Clamp
615 Hose Connector
617 Hose Clamp
620 Top Manifold Tube Inlet
640 Water pump manifold
642 Air Hose Line
643 Airline Connection
646 One Way Check Valve
648 Air Solenoid Valve
650 Air Hose Tubing Adapter Seal
660 90 Degree Tubing Connection
680 Inlet-Outlet Water Line
710 Bottom Water Manifold
711 Outlet water tube
712 Outlet water tube holes
715 Outlet Hose Straps
720 Top Water Manifold
722 Outlet Drain Hose
730 AC/DC Drain Pump
731 AC/DC water sensor switch
732 AC/DC Drain Pump wires
735 Pump Stand
740 Union for water line connections
742 Quick Disconnect for Outlet Hose
743 Hose Connection adapter
745 90 degree Pipe Fitting
747 4-Way pipe fitting
749 Water Outlet Port
751 Pipe fitting nipple
800 Hot Water Storage Tank Inlet and Lid
810 Cold Water Storage Tank Inlet and Lid
820 Cart Wheels
840 Cart Handle
860 AC 120-220 to DC Voltage converter
920 Cathode
940 Anode
960 Oxygen Line
980 Oxygen Separator Tube
1000 Hydrogen Gas Separator Tube
1020 Oxygen/Hydrogen Gas Chambers
1030 Hydrogen gas explosion vent hole
1040 Adjustable AMP DC Voltage transformer

The figures and the forgoing description give examples of embodiments. Those skilled in the art will appreciate that one or more of the described elements may well be combined into a single functional element. Alternatively, certain elements may be split into multiple functional elements. Elements from one embodiment may be added to another embodiment. For example, orders of processes described herein may be changed and are not limited to the manner described herein. Moreover, the actions of any flow diagram need not be implemented in the order shown; nor do all of the acts necessarily need to be performed. Also, those acts that are not dependent on other acts may be performed in parallel with the other acts. The scope of the embodiments is by no means limited by these specific examples. Numerous variations, whether explicitly given in the specification or not, such as differences in structure, dimension, and use of material, are possible.

1. A multilayered Hydrotherapy Bag, Hydrotherapy Bed, or Hydrotherapy Blanket for Dry hydrotherapy comprising:
at least three or more layers of waterproof fabrics and waterproof materials;
at least one cold water storage tank and at least one hot water storage tank configured to store cold and hot water respectively, for circulating inside the said Hydrotherapy Mummy Bag, Hydrotherapy Bag, Hydrotherapy Blanket or Hydrotherapy Bed;
at least one AC/DC water pump to circulate and drain the hot or cold water inside and outside the said Hydrotherapy Mummy Bag, Hydrotherapy Bag, Hydrotherapy Blanket and Hydrotherapy Bed;
at least one AC/DC water pump to drain the hot or cold water from inside the Hydrotherapy Mummy Bag, Hydrotherapy Bag, Hydrotherapy Blanket or Hydrotherapy Bed;
at least one insulated hot and cold water inlet tubing or pipe configured to separately circulate the hot and cold water inside the said Hydrotherapy Mummy Bag, Hydrotherapy Bag, Hydrotherapy Blanket and Hydrotherapy Bed;
at least one inlet line to the AC/DC water pump configured to circulate the hot and cold water inside the Hydrotherapy Mummy Bag, Hydrotherapy Bag, Hydrotherapy Blanket or Hydrotherapy Bed;
at least one AC/DC Two-Three-Way Solenoid Valve or Two-Three-Way Motorized Valve configured to circulate through the AC/DC water pump, cold water from the at least one cold water storage tank or hot water from the at least one hot water storage tank into the said Hydrotherapy Mummy Bag, Hydrotherapy Bag, Hydrotherapy Blanket and Hydrotherapy Bed;
at least one inlet water line for the AC/DC Two-Three-Way Solenoid Valve or AC/DC Two-Three-Way Motorized Valve, attached to the inlet line to the AC/DC water pump;
at least one air inlet line to an outer liner configured to deliver air to seal the said Hydrotherapy Mummy Bag, Hydrotherapy Bag, Hydrotherapy Blanket tightly around the body of the user;
at least one air compressor hose configured to attach to the water pump manifold configured to inject high-pressure air into water;
at least one AC/DC air compressor configured to compress air into the said Hydrotherapy Mummy Bag, Hydrotherapy Bag, Hydrotherapy Blanket and Hydrotherapy Bed;
plurality of Magnetic tubes configured to accommodate plurality of magnetic ball bearings and Ceramic Silicon Nitride ball bearings for providing massage;
plurality of plastic ball bearing separator configured to separate the magnetic field and to prevent sticking of magnetic bearings, when two or more magnetic bearings are used to increase the magnetic field inside the plurality of magnetic tubes;
plurality of Hydrotherapy tubes configured to accommodate hot or cold water for providing Hydrotherapy massage;
wherein the multilayered Hydrotherapy Mummy Bag, Hydrotherapy Bag, Hydrotherapy Blanket and Hydrotherapy Bed comprises a Transcutaneous Electrical Nerve Stimulation (TENS) device configured to stimulate the muscles and nerves;
wherein the water proofing element is Silicone or other Waterproof material configured to waterproof a Copper or Silver Infused Conductive Fabric;
wherein the TENS device comprises electrode connections, connected to a Copper or Silver Infused Conductive Fabric used to enhance the TENS device range;
wherein the multilayered Hydrotherapy Blanket, Hydrotherapy Mummy Bag, Hydrotherapy Bag or Hydrotherapy Bed further comprises:
at least one AC/DC air compressor;
at least one AC/DC water pump;
at least one DC supply from AC to DC voltage Converter configured to supply DC Volts to the Hydrotherapy Blanket, Hydrotherapy Mummy Bag, Hydrotherapy Bag or Hydrotherapy Bed electrical parts;
a positive DC wire that goes to a Cathode, and a negative DC wire that goes to an Anode;
at least one DC AMP Controller for the Cathode and Anode;
at least one Oxygen/Hydrogen Gas Chambers configured to hold water and an Electrolyte to produce pure Oxygen using the Anode for circulating the pure Oxygen inside the Hydrotherapy Blanket, Hydrotherapy Mummy Bag, Hydrotherapy Bag or Hydrotherapy Bed via at least one Oxygen line going from an Oxygen separator tube containing Oxygen from the Hydrogen gas;
plurality of Hydrotherapy nozzle holes configured to allow high pressured air and water to travel out of the Hydrotherapy tubes to massage the user's body without having the water touching the skin;
wherein the Hydrotherapy nozzle holes configured to allow high-pressure air and water to be injected from the Hydrotherapy tube nozzles onto the user's body to simulate a Hydro-therapeutic effect upon the skin and muscles of the user;
wherein the plurality of magnetic ball bearings inside the plurality of Magnetic tubes provide a magnetic field and as the plurality of magnetic ball bearings rolls down the magnetic tubes, the magnetic field travel across the user's body.

2. The Hydrotherapy Mummy Bag, Hydrotherapy Bag, Hydrotherapy Bed, or Hydrotherapy Blanket of claim 1, wherein material for at least three or more layers of the Hydrotherapy Mummy Bag, Hydrotherapy Bag, Hydrotherapy Bed, or Hydrotherapy Blanket is selected from either of Vinyl, Leather, Hemp fabric, waterproof Hemp fabric, waterproof Nylon, Neoprene, or other waterproof fabrics capable of withstanding high temperature and Pound per Square Inch (PSI).

3. The Hydrotherapy Mummy Bag, Hydrotherapy Bag, Hydrotherapy Bed, or Hydrotherapy Blanket of claim 1, comprises at least one AC/DC water heating element configured to heat the water in the at least one hot water storage tank.

4. The Hydrotherapy Mummy Bag, Hydrotherapy Bag, Hydrotherapy Bed, or Hydrotherapy Blanket of claim 1, comprises an Electrical storage box mounted on the at least one water storage tank for protecting at least one or more relay switches, breakers, timers, AC to DC Converters configured to control the opening and closing of the Solenoid Valves or Motorized Valves, operating the heater elements and water pumps.

5. The Hydrotherapy Mummy Bag, Hydrotherapy Bag, Hydrotherapy Bed, or Hydrotherapy Blanket of claim 1, comprises a zipper positioned on outer liner of the multilayered Hydrotherapy Bed configured to remove the water bed outer liner for washing or to repair or replace the outer liner.

6. The Hydrotherapy Mummy Bag, Hydrotherapy Bag, Hydrotherapy Bed, or Hydrotherapy Blanket of claim 1, wherein the AC/DC air compressor is configured to fill air inside of the Hydrotherapy Blanket, Hydrotherapy Mummy Bag, Hydrotherapy Bag or Hydrotherapy Bed to lift the user off a hard Surface, the High-PSI air is also used to compress the Hydrotherapy Blanket, Hydrotherapy Mummy Bag and Hydrotherapy Bag around the body part's tightly, and also the AC/DC air compressor's compressed air is used to inject into the Hydrotherapy nozzles to provide a Jacuzzi effect onto the body.

7. The Hydrotherapy Mummy Bag, Hydrotherapy Bag, Hydrotherapy Bed, or Hydrotherapy Blanket of claim 1, wherein the plurality of magnetic tubes or the plurality of Hydrotherapy tubes are high temperature tubing configured to circulate hot and cold water inside the Hydrotherapy Blanket, Hydrotherapy Mummy Bag, Hydrotherapy Bag or Hydrotherapy Bed.

8. The Hydrotherapy Mummy Bag, Hydrotherapy Bag, Hydrotherapy Bed, or Hydrotherapy Blanket of claim 1, comprises one way air check valve configured to direct the flow of air in one direction.

9. The Hydrotherapy Mummy Bag, Hydrotherapy Bag, Hydrotherapy Bed, or Hydrotherapy Blanket of claim 1, comprises at least one AC/DC timer and speed controller configured to switch on/off the water pump and to control water flow.

10. The Hydrotherapy Mummy Bag, Hydrotherapy Bag, Hydrotherapy Bed, or Hydrotherapy Blanket of claim 1, comprises at least one DC fuse and at least one AC electrical circuit breaker to prevent electrical shock.

11. The Hydrotherapy Mummy Bag, Hydrotherapy Bag, Hydrotherapy Bed, or Hydrotherapy Blanket of claim 1, comprises an AC/DC water heating element, thermostat sensor, and an AC/DC hot water a thermostat controller configured to heat and sense the water temperature and control the water temperature inside the at least one hot water storage tank.

12. The Hydrotherapy Mummy Bag, Hydrotherapy Bag, Hydrotherapy Bed, or Hydrotherapy Blanket of claim 1, comprises at least 1 AC/DC water level float to prevent damage to the AC/DC water pump and the other electrical equipment.

13. The Hydrotherapy Mummy Bag, Hydrotherapy Bag, Hydrotherapy Bed, or Hydrotherapy Blanket of claim 1, comprises magnetic sensor switches configured to stop the flow of water.

14. The Hydrotherapy Mummy Bag, Hydrotherapy Bag, Hydrotherapy Bed, or Hydrotherapy Blanket of claim 1, comprises TENS Device to massage.

15. The Hydrotherapy Mummy Bag, Hydrotherapy Bag, Hydrotherapy Bed, or Hydrotherapy Blanket of claim 1, comprises Oxygen Generator to supply Oxygen to Hydrotherapy Blanket, Hydrotherapy Mummy Bag, Hydrotherapy Bag or Hydrotherapy Bed.

16. The Hydrotherapy Mummy Bag, Hydrotherapy Bag, Hydrotherapy Bed, or Hydrotherapy Blanket of claim 1, comprises at least one AC/DC water pump to circulate hot or cold water. inside the Hydrotherapy Mummy Bag, Hydrotherapy Bag, Hydrotherapy Bed, or Hydrotherapy Blanket.

17. The Hydrotherapy Mummy Bag, Hydrotherapy Bag, Hydrotherapy Bed, or Hydrotherapy Blanket of claim 1, comprises at least one AC/DC water pump to drain the hot or cold water from inside the Hydrotherapy Mummy Bag, Hydrotherapy Bag, Hydrotherapy Bed, or Hydrotherapy Blanket.

18. The Hydrotherapy Mummy Bag, Hydrotherapy Bag, Hydrotherapy Bed, or Hydrotherapy Blanket of claim 1 is portable and can operate on DC Batteries, DC Solar Panels, or portable Wind Turbine Generators.

19. The Hydrotherapy Bag and Hydrotherapy Blanket of claim 1 is portable and can operate on at least 10 gallons of water/contaminated water to provide a safe Dry-hydrotherapy massage to the arm, leg or back.

20. The Hydrotherapy Mummy Bag, Hydrotherapy Bag, Hydrotherapy Blanket and Hydrotherapy Bed of claim 1 is portable and can be transported in a small vehicle for home to home dry hydrotherapy.
